(19) United States
(12) Patent Application Publication
(10) Pub. No.: US 2012/0035662 A1
(43) Pub. Date: Feb. 9, 2012

(54) ROD SCREW DYNAMIC IMPLANT FOR STABILIZING A VERTEBRAL COLUMN

(75) Inventors: Philippe Vincent-Prestigiacomo, Lacanau-Ocean (FR); Nicolas Roth, Bordeaux (FR)

(73) Assignee: CREASPINE, Pessac (FR)

(21) Appl. No.: 13/143,547

(22) PCT Filed: Jan. 5, 2010
(86) PCT No.: PCT/FR2010/050007

§ 371 Date: Oct. 3, 2011

(30) Foreign Application Priority Data
Jan. 7, 2009 (FR) 09/50068

(51) Int. Cl. A61B 17/70

(52) U.S. Cl. 606/264

(57) ABSTRACT

The invention relates to an implant for supporting a vertebral column, including at least one attachment element (300) and a connecting rod. Such an attachment element (300) includes an anchoring member (13) in a vertebral body, as well as a head (14) in which a passage opening (15) is provided that is conducive to receiving a connecting rod (12), the attachment element (300) being remarkable in that it comprises a means (316) for centering said rod, said rod-centering means being mounted on a spring-back means (317) placed in said head (14), said centering means (316) and said head (14) being movable relative to each other. A connecting rod (12) capable of being inserted into a passage opening (15) of an attachment element (300) includes at least one second centering surface (19). The attachment element (300) and the connecting rod (12) engage to form an implant in which said rod is rotatably and/or translatably movable relative to the anchoring member (13).

### TECHNICAL FIELD

The present invention relates to the field of vertebral column surgery on the human or animal body, and more particularly implants for stabilizing part of the vertebral column.

### BACKGROUND

The vertebral column plays a key role in many respects for the human or animal body and in particular, it must be able to absorb mechanical stresses due to movements of the body. The vertebral column may, however, no longer be able to correctly perform its functions, for example, following a trauma or for reasons related to age or illness that can cause wear, or degeneration of one or more vertebral articulations: these are referred to as instabilities. Such instabilities are very often a source of pain and can be treated using medication, rehabilitation, or surgery with or without instrumentalization.

The instrumentalization of the vertebral column consists of consolidating the stability of the vertebral column by placing an implant connecting the vertebral bodies of at least two consecutive vertebrae. Traditionally, such an implant comprises two elements connected to the vertebral bodies of the screw, hook, or cable type and are connected to each other by bar or plate-type elements. For clinical reasons, it is desirable for such an implant to have a certain flexibility and to absorb shocks or deform and avoid traumatizing the vertebral column. In this way, U.S. Pat. No. 6,966,910 describes an intervertebral bridge having two screws connected by a connection element whereof the geometric shape is conducive to bending in case of excessive stress. Such an intervertebral bridge does not, however, have all of the flexibility needed to avoid hindering the mobility of the part of the vertebral column thus treated. In order to impart greater mobility to the implanted part of a vertebral column, patent application US 2004/0049190 describes a dynamic device 1, shown in the appended FIG. 1, including two anchor elements 2 intended to be attached to vertebrae 3 connected by a bar 4 slidingly mounted to allow movement in the direction of the double arrow. An elastic means 5 is arranged on the bar 4, in the space 6 between the two anchor elements 2, symbolized by the square zone in broken lines, in order to exert a force in the direction of the longitudinal axis of the bar 4. Such a dynamic device 1 allows acceptable mobility of the treated part of the vertebral column, but is, however, bulky and cannot be used to correct all defects of the vertebral column. In fact, seen from the front or back, a healthy vertebral column is substantially rectilinear. However, in a sagittal plane, the vertebral column has four curves that, from bottom to top, are called sacral curve, lumbar lordosis, dorsal kyphosis and cervical lordosis. However, at the lumbar lordosis, the spaces between the vertebral bodies are particularly reduced and therefore a dynamic device 1 like that described in patent application U.S. 2004/0049190 cannot be positioned there due to the bulk of its elastic means 5. Furthermore, such a dynamic device 1 does not allow a dynamic control of the rotational movement of the bar 4 relative to the anchor elements 2.

### BRIEF SUMMARY

The present invention aims to resolve all or some of the aforementioned drawbacks. In this technical context, one aim of the present invention is to provide an implant to stabilize a vertebral column allowing translational and/or rotational mobility of the treated part of the vertebral column. More particularly, the present invention aims to obtain an implant able to be implanted at a lumbar lordosis or on part of a vertebral column whereof the vertebrae are not very spaced apart.

To that end, the present invention relates to an attachment element for a maintenance implant of part of a human or animal vertebral column, comprising, on the one hand, an anchor member intended to attach the element in a vertebral body and, on the other hand, a head in which a passage opening is formed conducive to receiving a connecting rod, this element being remarkable in that it includes means for centering said connecting rod mounted on a spring-back means arranged in said head, said centering means and said head being mobile relative to each other.

The present invention also relates to a connecting rod for a maintenance implant for a human or animal vertebral column, able to be inserted into a passage opening of an attachment element and comprising at least one second centering surface including a notch and/or a protrusion.

In this way, an attachment element according to the invention, in cooperation with a connecting rod according to the invention, makes it possible to obtain an implant according to the invention, as described below, offering suitable mobility and allowing dynamic control of the movement of the vertebrae of part of a vertebral column connected by said implant: in fact, during a relative movement of two vertebrae connected by an implant according to the invention, the connecting rod and the attachment element according to the invention can move translationally and/or rotationally relative to each other, said centering means tending to return the connecting rod and the attachment element in a predefined position, owing to the return forces exerted by the spring-back means. The spring-back means being arranged on the head of the attachment element, this allows the implant according to the invention to be positioned at a lumbar lordosis of a vertebral column: in fact, unlike a traditional device, it is not necessary to provide a space like the space usually required for the spring-back means of a dynamic device according to the prior art.

In one embodiment of the invention, the relative movement of the centering means is done along an axis transverse, in particular perpendicular, to the axis of the passage opening. An axis transverse to the axis of the opening makes it possible to reduce the minimum space needed between two vertebrae so they can be connected by an implant according to the present invention.

In one embodiment of the invention, said spring-back means tend to push said centering means towards the inside of the passage opening. The centering means pushed towards the inside of the passage opening are intended to bear on the connecting rod and minimize the functional play between the attachment element and said rod.

In one embodiment of the invention, said head also has a housing emerging in the passage opening inside which the spring-back means and the centering means are housed.

In one embodiment of the invention, said attachment element also comprising a bolt intended to close the housing made to emerge outside said head, the bolt comprising the spring-back means and the centering means. A bolt comprising the spring-back means and the centering means makes it possible to simplify the assembly of the attachment element.

In one embodiment of the invention, the bolt has flexible fins intended to cooperate with a stop formed in the surface of the housing. The fins are simple to make and can be molded with the bolt or can be machined on the bolt: this makes it possible to simplify the assembly, the number of pieces necessary for the assembly, and the production costs of the attachment element according to the invention.

In one embodiment of the invention, the centering means is secured to the anchor member, said spring-back means being mounted between the head and said anchor member. The spring-back means can then for example be formed by an inexpensive annular seal.

In one embodiment of the invention, the head and the anchor member are translationally and/or rotationally mobile relative to each other. Thus, an implant made using an attachment element according to the invention has increased mobility.

In one embodiment of the invention, the spring-back means is designed so as to exert a rotational return torque between the head and the anchor member. Thus, an attachment element according to the invention allows a dynamic control during translational and/or rotational movements of the vertebrae.

In one embodiment of the invention, said centering means includes a first centering surface opposite the passage opening having at least one slope, in particular planar or curved.

In one embodiment of the invention, the first centering surface has a notch.

In one embodiment of the invention, the first centering surface has a boss.

In one embodiment of the invention, at least part of the surface delimiting the passage opening has an additional centering surface having a boss and/or a notch.

In one embodiment of the connecting rod according to the invention, each second centering surface is formed near one of the ends of the connecting rod.

Lastly, the present invention relates to an implant for stabilizing a vertebral column in the human or animal body having at least one attachment element according to the invention and at least one connecting rod according to the invention inserted into the passage opening of said attachment element, the centering means being designed to cooperate with said second centering surface.

Preferably, said connecting rod is rotationally and/or translationally mobile relative to the anchor member, said first and second centering surfaces being designed to cooperate so as to move said connecting rod rotationally and/or translationally relative to the anchor member.

### BRIEF DESCRIPTION OF THE DRAWINGS

The invention will be better understood using the detailed description below in light of the appended drawings, in which:

FIG. 1 shows a diagrammatic view of a dynamic device according to the prior art, as mentioned in the preamble of the present description;

FIG. 2 shows a diagrammatic view of an implant according to the invention;

FIGS. 3a and 3b show longitudinal diagrammatic cross-sectional views of an attachment element according to a first embodiment of the invention and part of a connecting rod according to the invention;

FIGS. 4a to 4c show longitudinal diagrammatic cross-sectional views of an attachment element according to a second embodiment of the invention and part of a connecting rod according to the invention;

FIGS. 5a and 5b respectively show longitudinal diagrammatic cross-sectional views of an attachment element according to a third embodiment of the invention and part of a connecting rod according to the invention;

FIGS. 6a to 6c show perspective (6a, 6b) and longitudinal cross-sectional (6c) diagrammatic views of an attachment element according to a fourth embodiment of the invention and part of a connecting rod according to the invention;

FIGS. 7a to 7f show diagrammatic perspective views of details of second centering surfaces of the connecting rod according to the invention.

### DETAILED DESCRIPTION

An implant 10 according to the invention, as shown diagrammatically in

FIG. 2, has at least one attachment element 11 according to the invention intended to be anchored in a vertebra 3 and at least one connecting rod 12 according to the invention. The attachment element 11 includes an anchor member 13 intended to attach said attachment element 11 in the vertebral body of a vertebra 3. The attachment element 11 also has a head 14 in which a passage opening 15 is formed conducive to receiving the connecting rod 12, so that the latter can slide inside said passage opening 15 at least in the direction symbolized by the double arrow. In the head 14 of the attachment element 11, a means 16 for centering the connecting rod 12 is arranged mounted on a spring-back means 17. The centering means 16 has a first centering surface 18 opposite the passage opening 15 and having at least one planar or curved slope, said first centering surface 18 forming a boss and/or a notch. The spring-back means 17 exerts forces on the centering means 16 so as to push said first centering surface 18 towards the inside of the passage opening 15.

The connecting rod 12 has, at one of its ends intended to be inserted into the passage opening 15, a second centering surface 19 also having a notch and/or a protrusion. At the other end of the connecting rod 12, a traditional anchor screw 20 is for example attached, intended to attach the implant 10 according to the invention in a second vertebra 3.

When the connecting rod 12 is inserted into the passage opening 15, the first and second centering surfaces (18, 19) cooperate, as detailed later, to move said connecting rod 12, rotationally and/or translationally, relative to the anchor member 13.

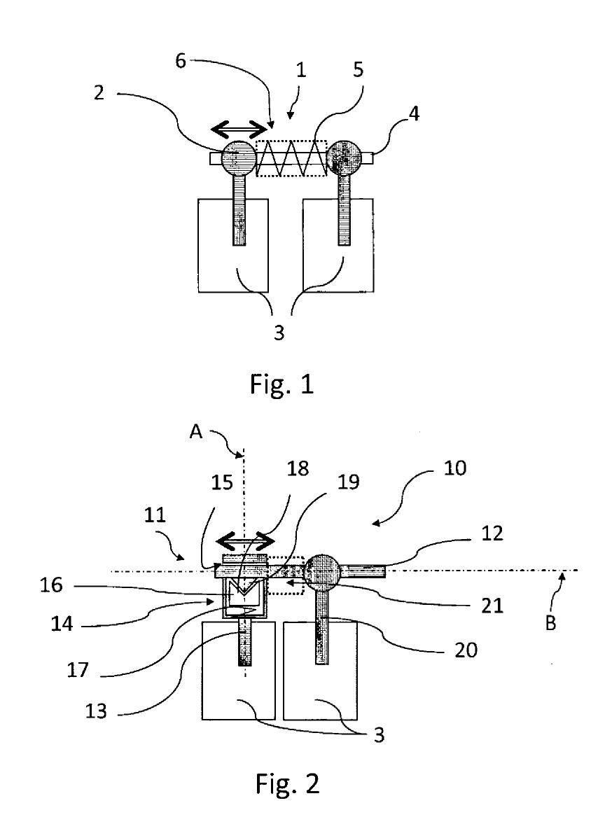

Incorporating a spring-back means 17 inside the head 14 makes it possible to free the space occupied by the spring-back means 5 on the bar 4 of a traditional dynamic device 1. In this way, the space 21, symbolized by the square zone in dotted lines in FIG. 2, situated between the fastening element 11 and the anchor screw 20 of the implant 10 of FIG. 2, is more limited than the space 6 occupied by the elastic means 5 of a traditional dynamic device 1 shown in FIG. 1. Moreover, the relative movement of the centering means 16 along an axis A transverse to the axis B of the passage opening 15 makes it possible to position the centering means 16 outside the space 21 and therefore to further reduce the latter. The bulk of the space 21 by the centering means 16 is minimal when the transverse axis A is perpendicular to the axis B of the passage opening 15.

Such a limited space 21 makes it possible to position an implant 10 according to the invention on two consecutive vertebrae 3 situated in a zone of the vertebral column, such as the lumbar lordosis, when a traditional dynamic device 1 cannot be positioned because it is too bulky.

In the continuation of the description, and in order to simplify the reading thereof, elements similar or identical to the various attachment elements may keep the same numbering.

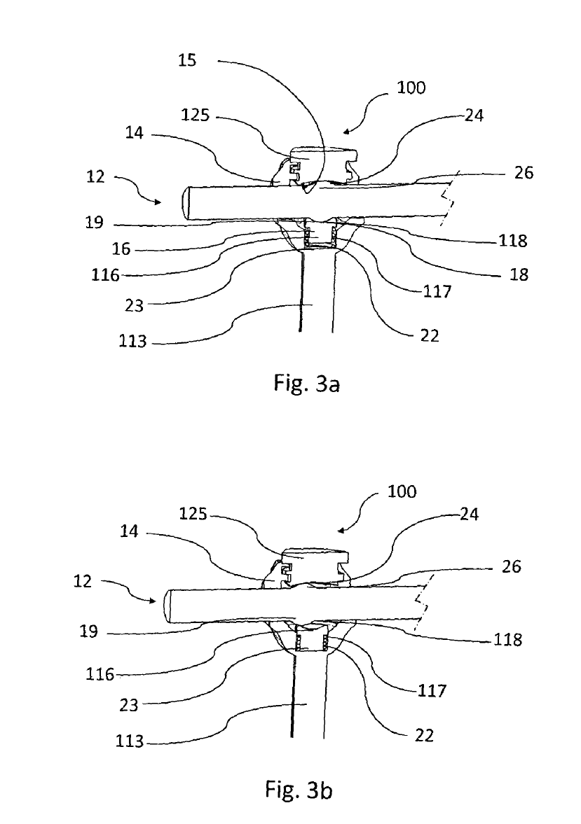

FIGS. 3a and 3b are longitudinal cross-sectional views of an attachment element 100 according to a first embodiment of the invention, in the passage opening 15 in which a connecting rod 12 according to the invention is inserted. The attachment element 100 has a head 14 integral with the anchor member 13 for example formed by a screw 113. In this embodiment, the head 14 has a housing 22, emerging inside the passage opening 15, in which the spring-back means 17, in this case formed by a helical spring 117, and the centering means 17 then made up of a centering block 116 mounted on the helical spring 117, on the other hand, are arranged. The centering block 116 can move along an axis parallel to the longitudinal axis of the screw 113 and the helical spring 117 pushes the centering block 116 towards the inside of the passage opening 15 by freeing a space 23 between the centering block 116 and the bottom of the housing 22.

The centering block 116 has a first centering surface 18, for example a spherical notch 118, situated opposite the passage opening 15. Advantageously, an additional centering surface 24 delimiting the passage opening 15, and opposite the first centering surface 18, also has a boss or a notch. In the embodiment of the invention shown in FIG. 3a, the recess 22 extends through the passage opening 15 and emerges outside the head 14. Such a recess 22 is easy to produce and can be closed by a bolt 125. The additional centering surface 24 is, for example, the face of a bolt 125 intended to arrive opposite the passage opening 15.

The connecting rod 12 according to the invention has a second centering surface 19 shown in this embodiment by a rounded boss 26. The first and second centering surfaces (18, 19) are kept in contact owing to the helical spring 117. In the absence of stress of the connecting rod 12 and/or the attachment element 100, and owing to the complementary shapes of the rounded boss 26 and the spherical notch 118, the connecting rod 12 occupies a predefined position as shown in FIG. 3a.

When a force is applied on the implant 10 according to the invention, for example when the vertebrae 3 supporting the implant 10 move relative to each other, the connecting rod 12 and the attachment element 100 can be made to move relative to each other. During such movement, the first and second centering surfaces (18, 19) slide one on the other and are kept in contact owing to the pressure exerted by the helical spring 117. During the translational movement of the connecting rod 12 in the passage opening 15, the rounded boss 26 forces the centering block 116 into a translational movement in the housing 22. In this way, when the connecting rod 12 moves away from the predefined position shown in FIG. 3a, the rounded boss 26, given the shape of the spherical notch 118, pushes the centering block 116 so as to compress the helical spring 117 by reducing the space 23 between the centering block 116 and the bottom of the housing 22, as shown in FIG. 3b. The compression of the helical spring 117 then opposes, in this case, the relative movement of the connecting rod 12 in the passage opening 15. Thus, when the connecting rod 12 and/or the attachment element 100 are no longer long subjected to an outside force involving their relative movement, as the first and second centering surfaces (18, 19) are in contact, these sliding one on the other so as to relax the helical spring 117. Such a sliding results in returning the connecting rod 12 towards the predefined position shown in FIG. 3a.

The centering block 116, pushed by the helical spring 117, exerts a force on the connecting rod 12, which is kept pressed against the additional centering surface 24. This additional centering surface 24, in the same way as the first centering surface 18, participates in guiding the connecting rod 12 towards the predefined position of FIG. 3a, owing to its adapted shape complementary to the rounded boss 26.

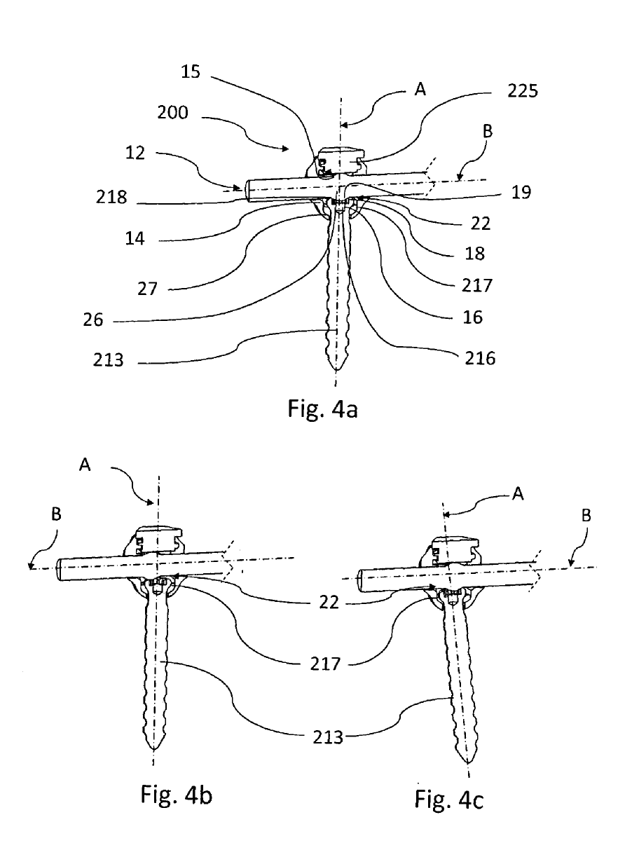

FIGS. 4a, 4b and 4c are longitudinal cross-sectional views of an attachment element 200 according to a second embodiment of the invention, in the passage opening 15 in which a connecting rod 12 is introduced similar to that already described in FIGS. 3a and 3b, for example. The attachment element 200 has a head 14 and an anchor member 13, for example comprising a screw 213, able to move translationally and/or rotationally relative to each other. In this embodiment, the head 14 has a housing 22, emerging inside the passage opening 15. In this embodiment, the housing 22 is a through housing, along an axis A substantially perpendicular to the axis B of the passage opening 15. One of the openings of the housing 22 has a rim 27 and the other opening is intended to be closed by a bolt 225. In this attachment element 200, the centering means 16 is secured to the anchor member 13 and forms one of the ends 216 of said screw 213. The anchor member 13 and the rim 27 are designed so that the screw 213 can extend through the opening of the housing 22 including said rim 27 and so as to prevent the passage of the centering means 16. According to this embodiment, the spring-back means 17 has an elastically deformable annular seal 217 arranged inside the housing 22 and resting on the rim 27, the annular seal 217 being arranged to be inserted between said rim 27 and the centering means 16. Advantageously, the annular seal 217 is arranged to exert a rotational return torque between the anchor member 13 and the head 14, towards an aligned position of these two members along the transverse axis A: such a return torque is due to the elasticity of the seal 217, stuck between the housing 22 on the one hand and the centering means 16 on the other hand. The annular seal 217 being elastically deformable, it also allows, in addition to a rotational movement, a translational movement of the end 216 of the screw 213 relative to the head 14 when it undergoes elastic crushing.

The end 216 comprises a first centering surface 18 formed, according to this embodiment, by a centering rim 218 formed at the end 216 of the anchor member 13 intended to be opposite the passage opening 15. When the connecting rod 12 is introduced into the passage opening 15, it can occupy a predefined and privileged position, as shown in FIG. 4a, in which the centering rim 218 and the rounded boss 26 are in contact, and in which the walls of the passage opening 15 and/or the end of the bolt 215 opposite the passage opening 15 are also in contact with the rounded boss 26. In this predefined position, the annular joint 217 does not undergo dissymmetrical crushing relative to the axis A.

In the case of a relative movement of the connecting rod 12 and the attachment element 200, the rounded boss 26 in contact with the centering rim 218 imposes an elastic deformation on the annular seal 217, visible in particular in FIG. 4c, which creates an elastic effort tending to return the connecting rod 12 and the attachment element 200 to a predefined position as described in Figure 4a. The annular seal 217 has the advantage of allowing a great freedom of relative movement between the head 14, the screw 213, and the connecting rod 12, rotationally and/or translationally. In particular, the annular seal 217 allows one of the screw 213 and the connecting rod 12 as shown in FIG. 4c via the axes A and B.

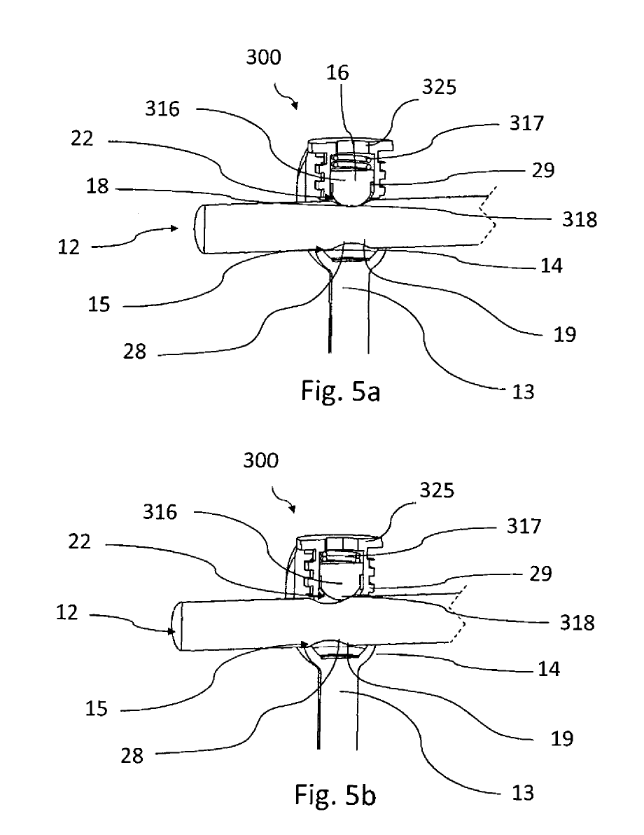

According to a third embodiment, as shown in FIGS. 5a and 5b, an attachment element 300 differs from the attachment element 100 in that a housing 29 is formed in a bolt 325, said housing forming a continuation of the housing 22 that the bolt 325 is meant to close. The bolt 325 is, for example, attached to the head 14 using a screw pitch formed in the wall of the housing 22. This housing 29 receives a helical spring 317 and a centering block 316 mounted on said helical spring 317, the centering block 316 protruding from said housing 22 into the passage opening 15, when the bolt 325 is positioned in the housing 22. This centering block 316, like the centering block 116 of the first embodiment described in FIGS. 3a and 3b, constitutes the centering means 16. The first centering surface 18 making up the face of the centering block 316 opposite the passage opening 15 is for example a rounded boss 318. In that case, the second centering surface 19 formed in the connecting rod 12 adapted to said attachment element 300 is a rounded notch 28. As for the attachment element 100 of the first embodiment, a relative movement of the connecting rod 12 relative to the head 14 of the attachment element 300 involves a compression of the helical spring 317, as shown in FIG. 5b, which then exerts a force on the centering block 316, transmitted to the connecting rod 12 via the sliding contact between the first centering surface 18 and the second centering surface 19. This force tends to return the connecting rod 12 to the predefined position as shown in FIG. 5a.

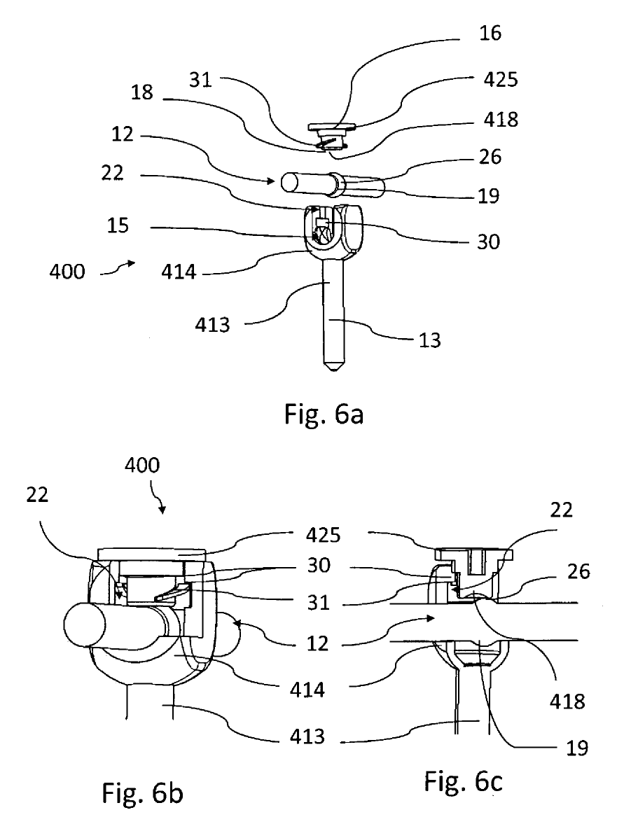

According to a fourth embodiment, like that shown in FIGS. 6a to 6c, an attachment element 400 has an anchor member 13 made up of a screw 413 extended by a U-shaped head 414, the space situated between the branches of the “U” constituting the housing 22 and the passage opening 15. In this embodiment, the housing 22 has at least one stop 30 formed in the surface of said housing 22, i.e. on the branches of the “U.” The attachment element 400, shown in FIGS. 6a to 6c, has two stops 30 arranged opposite each other and protruding in the housing 22. According to an alternative of the invention that is not shown, a stop is for example a screw pitch portion.

A bolt 425 intended to close the housing 22 has at least two flexible fins 31 intended to cooperate with said stops 30 in order to allow the bolt 425 to be fastened on the head 414. The bolt 425 has a first centering surface 18 formed by a rounded notch 418, visible in FIG. 6c, situated on the face of the bolt 425 intended to be placed opposite the passage opening 15.

The assembly of the implant 10 done using an attachment element 400 is particularly easy since a connecting rod 12, like that previously described with the first and second embodiments of the attachment element (100, 200), is inserted between the branches of the U-shaped head 414, as shown in FIG. 6a, then the bolt 425 is screwed on the head 414 to make the fins 31 cooperate with the stops 30 in order to retain the connecting rod 12. Once the bolt 425 is positioned and retained by the fins 31 and the stops 30, the first and second centering surface (18, 19) ensure the centering using the fins 31. The latter parts act as spring-back means 16 owing to their flexibility, in addition to their fastening function of the bolt 425. Thus, upon a relative movement of the connecting rod 12 in relation to the attachment element 400, the sliding of the rounded boss 26 along the rounded notch 418 causes a movement of the bolt 425. This movement of the bolt is done along an axis parallel to the branches of the “U” of the head 414 owing to an elastic deformation of the fins 31, as shown in FIG. 6c. These elastic deformations of the fins 31 create a spring-back force opposing the movement of said bolt 425 and the connecting rod 12, tending to return said connecting rod 12 by sliding of the first and second centering surfaces (18, 19) into a predefined position described in FIGS. 6a and 6b. In this predefined position, the fins 31 do not undergo any elastic deformation.

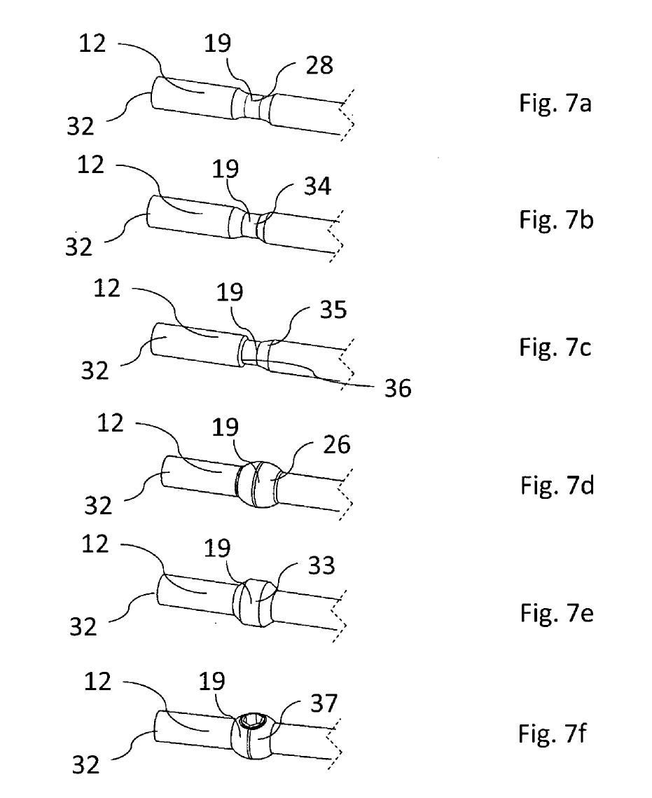

The connecting rod 12 according to the invention, used in cooperation with an attachment element 100, 200, 300, 400 according to the invention, has, near one 32 of its ends, a second centering surface 19 such as a rounded boss 26, described in FIGS. 3a, 3b, 4a, 4b, 4c, 6a, 6b and 6c, or a rounded notch 28 as shown in FIGS. 5a and 5b. The second centering surface 19 is not, however, limited to these shapes and can, for example, assume various shapes like those shown in FIGS. 7a to 7f. One skilled in the art will then know how to adapt the shape of the first centering surface to ensure adequate cooperation between the connecting rod and the attachment element. This results in the rounded boss 26 shown in FIG. 7d and the rounded notch 28 of FIG. 7a. A boss can also have non-curved slopes such as, for example, the boss 33 of FIG. 7e. Likewise, a notch can have non-rounded slopes like those of the notch 34 shown in FIG. 7b.

Moreover, the notch or the boss can be asymmetrical like the notch 35 shown in FIG. 7c, which has a wall 36 forming a stop. Such a notch 35 can be interesting to prevent the relative movement in a direction of the connecting rod 12 with the associated attachment element, owing to the wall 36 forming the stop: the wall 36 forming the stop prevents the movement of the attachment element towards the end 32 of said connecting rod 12.

Furthermore, the second centering surface 19 can also be formed by a ring 37 attached on the connecting rod 12 according to the invention as is the case of the connecting rod 12 shown in FIG. 7f.

Of course, the embodiments mentioned above are in no way limiting and other details and improvements can be made to an attachment element 100, 200, 300, 400 or to a connecting rod 12 according to the invention, without going beyond the scope of the invention where other shapes of an attachment element or a connecting rod can be made: in particular, aside from the shapes detailed in the Figures, the first and second centering surfaces (18, 19) can include rotational centering elements, not shown, able to impart a rotational movement so as to return the connecting rod to a predefined position.

1. An attachment element for a maintenance implant of part of a human or animal vertebral column, comprising;
an anchor member intended to attach the element in a vertebral body,
a head in which a passage opening is formed conducive to receiving a connecting rod, and
a means for centering said connecting rod mounted on a spring-back means arranged in said head,
wherein said centering means and said head being mobile relative to each other.

2. The attachment element according to claim 1, wherein relative movement of the centering means is done along an axis transverse, to an axis of the passage opening.

3. The attachment element according to claim 1, wherein said spring-back means push said centering means towards an inside of the passage opening.

4. The attachment element according to claim 1, wherein said head also has a housing emerging in the passage opening inside which the spring-back means and the centering means are housed.

5. The attachment element according to claim 2, also comprising a bolt intended to close the housing made to emerge outside said head, the bolt comprising the spring-back means and the centering means.

6. The attachment element according to claim 2, wherein the bolt has flexible fins intended to cooperate with a stop formed in the surface of the housing.

7. The attachment element according to claim 2, wherein the centering means is secured to the anchor member, said spring-back means being mounted between the head and said anchor member.

8. The attachment element according to claim 7, wherein the head and the anchor member are translationally and/or rotationally mobile relative to each other.

9. The attachment element according to claim 8, wherein the spring-back means is designed so as to exert a rotational return torque between the head and the anchor member.

10. The attachment element according to claim 1, wherein said centering means includes a first centering surface opposite the passage opening having at least one slope, in particular planar or curved.

11. The attachment element according to claim 10, wherein the first centering surface has a notch.

12. The attachment element according to claim 10, wherein the first centering surface has a boss.

13. The attachment element according to claim 1, wherein at least part of a surface delimiting the passage opening has an additional centering surface having a boss and/or a notch.

14. A connecting rod for a maintenance implant for a human or animal vertebral column, able to be inserted into a passage opening of an attachment element according to claim 1, and comprising:
at least one second centering surface, including a notch and/or a protrusion, said second surface being able to cooperate with the first centering surface of the centering means.

15. The connecting rod according to claim 14, wherein each second surface is formed near one end of the connecting rod.

16. An implant for stabilizing a vertebral column in the human or animal body having at least one attachment element and at least one connecting rod according to claim 14, inserted into the passage opening of said attachment element, the centering means being designed to cooperate with said second centering surface.

17. The implant according to claim 16, wherein said centering means includes a first centering surface opposite the passage opening having at least one slope, in particular planar or curved, and wherein said connecting rod is rotationally and/or translationally mobile relative to the anchor member, said first and second centering surfaces being designed to cooperate so as to move said connecting rod rotationally and/or translationally relative to the anchor member.
